(19) United States
(12) Patent Application Publication
(10) Pub. No.: US 2024/0208892 A1
(43) Pub. Date: Jun. 27, 2024

(54) EXTRACTION METHOD

(71) Applicant: BASF SE, Ludwigshafen am Rhein (DE)

(72) Inventors: Edward RICHMOND, Ludwigshafen am Rhein (DE); Wolf-Steffen WEISSKER, Ludwigshafen am Rhein (DE); Rocco PACIELLO, Ludwigshafen am Rhein (DE)

(21) Appl. No.: 18/287,503

(22) PCT Filed: Apr. 11, 2022
(86) PCT No.: PCT/EP2022/059568

§ 371 Date: Oct. 19, 2023

(30) Foreign Application Priority Data
Apr. 22, 2021 (EP) 21169900.4

(51) Int. Cl. C07C 51/48; B01D 11/04; C07C 51/15

(52) U.S. Cl. CPC C07C 51/48; B01D 11/0492; C07C 51/15

(57) ABSTRACT

A method for separating a salt S, wherein the salt S is present dispersed in an aprotic polar liquid A, the liquid A containing the dispersed salt S is extracted with a non-polar liquid B, wherein the salt S is dispersed in the liquid B, and the liquid B containing the dispersed salt S is extracted with water, wherein the solid is dissolved in water.

The present invention relates to a method for separating a salt S, wherein the salt S is present dispersed in an aprotic polar liquid A, the liquid A containing the dispersed salt S is extracted with a non-polar liquid B, wherein the salt S is dispersed in the liquid B, and the liquid B containing the dispersed salt S is extracted with water, wherein the solid is dissolved in water.

The older PCT application with the file number PCT/EP2021/056750 describes a method for preparing salts of α,β-unsaturated carboxylic acids from ethene and carbon dioxide. The salt is finely dispersed in an organic solvent. The organic solvents used are miscible with water. Therefore, the salt has to be separated by filtration. The disadvantage of the method is that the finely dispersed salt is difficult to filter.

The object was therefore to find an alternative method for separating finely dispersed salts from an organic solvent.

The object is achieved by a method for separating a salt S, wherein the salt S is present dispersed in an aprotic polar liquid A, wherein
(a) the liquid A containing the dispersed salt S is extracted with a non-polar liquid B, wherein the salt S is dispersed in the liquid B, and
(b) the liquid B containing the dispersed salt S is extracted with water, wherein the solid is dissolved in water,
   wherein the solubility of liquid A in liquid B at 25° C. is less than 20% by weight and the solubility of the salt S in water at 25° C. is at least 20% by weight.

Surprisingly, it has been found that when extracted with a non-polar solvent, finely dispersed salt passes into the non-polar solvent in the aprotic polar solvent. It is possible that the finely dispersed salt disrupts the dipole-dipole interactions of the solvent molecules of the aprotic polar solvent.

The salt S is preferably an organic salt, particularly preferably an acrylic acid salt, especially preferably sodium acrylate.

Suitable organic salts are, for example, alkali metal salts of carboxylic acids, for example sodium acetate, sodium propionate, sodium acrylate, potassium acrylate and tripotassium citrate.

The amount of water in step (b) is selected such that an aqueous solution of an acrylic acid salt is obtained that is preferably at least 25% by weight, particularly preferably at least 30% by weight, especially preferably at least 35% by weight.

An aprotic polar liquid A does not comprise any heteroatom-hydrogen bonds such as nitrogen-hydrogen bonds and oxygen-hydrogen bonds.

Suitable aprotic polar liquids A are, for example, dimethylformamide, sulfolane, dimethyl sulfoxide, propylene carbonate, nitromethane, nitrobenzene, benzonitrile or mixtures thereof. The liquid A should comprise less than 1% by weight water.

The aprotic polar liquid A may additionally comprise a secondary or tertiary alkanol, for example 3,7-dimethyloctan-3-ol. These alkanols are used as auxiliaries in the production of acrylic acid salts from ethene and carbon dioxide. The alkanol content in the liquid A is usually from 5 to 15% by weight.

Suitable non-polar liquids B are, for example, alkanes, alkenes, aromatics, trialkylamines, dialkyl ethers or mixtures thereof.

The salt S should have an average particle size of 3 to 30 μm and/or a particle size distribution width of less than 3.00. The average particle size is measured by laser diffraction, the average particle size being the volume-averaged average particle size. The width of the particle size distribution is determined using the cumulative distribution curve according to (d₉₀-d₁₀)/(2×d₅₀), where d₁₀ is the cumulative 10% particle size and doo is the cumulative 90% particle size and d₅₀ is the average particle size.

The particles can also be secondary particles (agglomerates) made up of smaller primary particles of less than 1 μm.

The extractions in steps (a) and (b) are preferably carried out at a temperature of 10 to 60° C. Lower temperatures increase the miscibility gap of the solvents used. Higher temperatures require less cooling.

The ratio of liquid A to liquid B in step (a) is preferably from 1 to 10, particularly preferably from 0.2 to 5, especially preferably from 0.5 to 2. Phase separation is facilitated by a phase ratio in these ranges.

The ratio of liquid B to water in step (b) is preferably from 1 to 100, particularly preferably from 2 to 50, especially preferably from 5 to 20. A phase ratio in these ranges facilitates the preparation of concentrated aqueous solutions of the salt S.

The present invention further relates to a method for the preparation of acrylic acid salts, comprising
(i) reacting ethene and carbon dioxide in an aprotic polar liquid A in the presence of a carboxylation catalyst and a secondary or tertiary alkoxide to obtain an acrylic acid salt dispersed in the liquid A and
(ii) separating the dispersed acrylic acid salt according to the method according to the invention,
   and the liquid A and the liquid B are recycled to the process.

The ethene partial pressure in step (i) is preferably from 0.5 to 100 bar, particularly preferably from 2 to 80 bar, especially preferably from 5 to 50 bar.

The carbon dioxide in step (i) can be used in gaseous, liquid or supercritical form. It is also possible to use gas mixtures comprising carbon dioxide on an industrial scale, provided they do not comprise any appreciable amounts of carbon monoxide.

The carbon dioxide partial pressure in step (i) is preferably from 1 to 200 bar, particularly preferably from 4 to 140 bar, especially preferably from 10 to 100 bar.

The molar ratio of carbon dioxide to ethene is preferably from 0.1 to 15, particularly preferably from 1 to 10, especially preferably from 4 to 8.

Other inert gases such as nitrogen and noble gases may be present. However, the proportion thereof should be less than 10 mol %.

Secondary or tertiary alkoxides are based on secondary or tertiary alkanols. Secondary or tertiary alkanols are alkanols where the hydroxyl group is on a secondary or tertiary carbon atom.

Suitable alkoxides are sodium propan-2-olate, sodium tert-butoxide, sodium cyclopentanolate, sodium cyclohexanolate, sodium cycloheptanolate, sodium butan-2-olate, sodium 3-methylbutan-2-olate, sodium 4-methylbutan-2-olate, sodium pentan-3-olate, sodium 1-methoxypropan-2-olate, sodium 1-methylcyclopentan-1-olate, sodium 1-methylcyclohexan-1-olate, sodium 2-phenylpropan-2-olate, sodium 3-methyheptan-3-olate, sodium 3-methyhexan-3-olate, sodium 2-methylhexan-2-olate, sodium 2-methylbutan-2-olate, sodium 3-ethylpentan-3-olate, sodium 2-methylpentan-2-olate, sodium 3-methylpentan-3-olate, sodium 3,7-dimethyloctan-3-olate, sodium 2,3-dimethylbutan-2-olate or mixtures thereof.

The secondary or tertiary alkoxide is consumed stoichiometrically. Here, the corresponding alkanol is formed. The secondary or tertiary alkanol can be converted to the corresponding alkoxide with sodium methoxide.

In step (i), ethene and carbon dioxide are reacted in the presence of a carboxylation catalyst. Transition metal complexes are typically used as carboxylation catalysts. The carboxylation catalysts are used in an amount of preferably 0.1 to 20 000 ppm by weight, particularly preferably from 1 to 1000 ppm by weight, especially preferably from 5 to 500 ppm by weight, based in each case on the reaction mixture. Suitable transition metals are the transition metals of groups 4, 6, 7, 8, 9 and 10 of the periodic table of the elements. Preference is given to nickel and palladium. Particular preference is given to palladium.

Phosphorus-based bidentate ligands are advantageously used as ligands of the transition metal complexes. Suitable ligands are 1,2-bis(dicyclohexylphosphino)ethane, 2,3-bis(dicyclohexylphosphino)butane, 1,2-bis(diisopropylphosphino)ethane, 1,2-bis(dodecylphosphino)ethane, 1,2-bis(di-tert-butylphosphino)ethane, 1,2-bis(dicyclopentylphosphino)ethane, 1,2-bis(dicyclohexylphosphino)cyclohexane and mixtures thereof.

The transition metal complex can be prepared directly from transition metal in oxidation state 0 and ligand. However, it is also possible to first produce a precursor of the transition metal complex and then to reduce it. Suitable reducing agents are hydrogen, magnesium, sodium and zinc.

Suitable precursors of the transition metal complex are bis(cycloocta-1,5-diene)nickel, bis(acetylacetone)nickel, tetrakis(triphenylphosphine)nickel, bis(dibenzylideneacetone)palladium, tris(dibenzylideneacetone)dipalladium, tetrakis(triphenylphosphine)palladium, cyclopentadienyl allyl palladium, cyclopentadienyl cinnamyl palladium or mixtures thereof.

The reaction in step (i) is carried out at a temperature of preferably 20 to 250° C., particularly preferably 50 to 190° C., especially preferably 70 to 180ºC. The total pressure is preferably from 1 to 300 bar, particularly preferably 3 to 200 bar, especially preferably 5 to 150 bar.

The reaction in step (i) can be carried out in standard reactors suitable for gaseous/liquid reactions. Such reactors are described, for example, in S. Moran, K.-D. Henkel “Reactor Types and Their Industrial Application”, Chapter 3.3 “Reactors for gas-liquid reactions” (Ullmann's Encyclopedia of Industrial Chemistry, Wiley VCH Verlag Gmbh & Co KGaA, DOI: 10.1002/14356007.b04_087).

The aprotic polar liquid A separated off in step (ii) during extraction (a) can be recycled to step (i), optionally after purification and removal of the alkanol and water used as auxiliary.

The non-polar liquid B separated off in step (ii) during extraction (b) can be recycled to the extraction (a), optionally after purification and removal of the alkanol and water used as auxiliary.

The resulting aqueous solution of the acrylic acid salt can be purified, for example by filtration using activated charcoal, stripping with steam or distillation. Residues of the carboxylation catalyst can be removed with an ion exchanger.

The aqueous solution of the acrylic acid salt is suitable for producing soluble or water-swellable polyacrylic acid salts, especially excessively weakly crosslinked polyacrylic acid salts (super-absorbers).

### EXAMPLES

4.63 g (4.00 mmol) of tetrakis(triphenylphosphine)palladium, 1.87 g (4.40 mmol) of 1,2-bis(dicyclohexylphosphino)ethane, 72.1 g (400.0 mmol) of sodium 3,7-dimethyloctan-3-olate, 16.9 g (107 mmol) of 3,7-dimethyloctan-3-ol and 600 ml of dimethylformamide (liquid A) were initially charged in a 3.5 l autoclave under an argon atmosphere. The starting materials were dissolved by stirring at 500 rpm for 15 minutes. Carbon dioxide (330 g, 7.50 mol) and ethene (33.0 g, 1.18 mol) were then introduced under pressure at 25° C. The mixture was then stirred at 145° C. and a total pressure of 83 bar at 750 rpm for 2 hours. After cooling to 50° C., the pressure was released. The autoclave was emptied into a 1 l glass flask and rinsed with 150 ml of dimethylformamide. A dispersion of sodium acrylate was obtained. The mean particle size of the agglomerates was 7.8 μm, the width of the particle size distribution was 1.65.

The dispersion obtained was extracted at a 1:1 ratio with a non-polar solvent (liquid B). The dispersed sodium acrylate passed into the non-polar solvent. The non-polar solvent was then extracted with 70 ml of water and the aqueous sodium acrylate obtained was treated with activated charcoal. A pale yellowish solution was obtained. The extractions were carried out at a temperature of 23° C.

The non-polar solvents (liquid B) used were respectively n-pentane, n-hexane, hexane mixtures, n-heptane, n-octane, isooctane, n-nonane, n-decane, n-undecane, n-dodecane, tributylamine, trihexylamine, trioctylamine and tridodecylamine.

In the extractions with n-nonane and trioctylamine, prior to the extraction with 70 ml of water, dimethylformamide present in the non-polar solvent was distilled off.

1.-15. (canceled)

16. A method for separating a salt S, wherein the salt S is present dispersed in an aprotic polar liquid A, wherein
(a) the liquid A containing the dispersed salt S is extracted with a non-polar liquid B, wherein the salt S is dispersed in the liquid B, and
(b) the liquid B containing the dispersed salt S is extracted with water, wherein the solid is dissolved in water,
wherein the solubility of liquid A in liquid B at 25° C. is less than 20% by weight and the solubility of the salt S in water at 25° C. is at least 20% by weight.

17. The method according to claim 16, wherein the salt S is an organic salt.

18. The method according to claim 16, wherein the salt S is an acrylic acid salt.

19. The method according to claim 18, wherein an at least 25% by weight aqueous solution of the acrylic acid salt is obtained in step (b).

20. The method according to claim 16, wherein the liquid A is dimethylformamide.

21. The method according to claim 16, wherein the liquid B is a C₅- to C₁₂-alkane or a tri-(C₄- to C₁₂-alkyl)amine.

22. The method according to claim 16, wherein the liquid A comprises less than 1% by weight water.

23. The method according to claim 16, wherein the salt S has an average particle size of 3 to 30 μm.

24. The method according to claim 16, wherein the salt S has a particle size distribution width of less than 3.00.

25. The method according to claim 16, wherein the extractions are carried out at a temperature of 10° C. to 60° C.

26. The method according to claim 16, wherein the ratio of liquid A to liquid B in step (a) is from 0.2 to 5.

27. The method according to claim 16, wherein the ratio of liquid B to water in step (b) is from 5 to 20.

28. The method according to claim 16, wherein the liquid A comprises a secondary or tertiary alkanol.

29. The method according to claim 28, wherein the alkanol is a tertiary C₈- to C₁₂-alkanol.

30. A method for preparing acrylic acid salts, comprising
(i) reacting ethene and carbon dioxide in an aprotic polar liquid A in the presence of a carboxylation catalyst and a secondary or tertiary alkoxide to obtain an acrylic acid salt dispersed in the liquid A and
(ii) separating the dispersed acrylic acid salt according to a method of claim 16,
and the liquid A and the liquid B are recycled to the process.
